(19) United States
(12) Patent Application Publication
(10) Pub. No.: US 2007/0128076 A1
(43) Pub. Date: Jun. 7, 2007

(54) ELECTRICAL TEST APPARATUS

(76) Inventor: Gunther Bohm, Nufringen (DE)

Correspondence Address: OSTROLENK FABER GERB & SOFFEN, 1180 AVENUE OF THE AMERICAS, NEW YORK, NY, 100368403, US

(21) Appl. No.: 11/566,979

(22) Filed: Dec. 5, 2006

(30) Foreign Application Priority Data
Nov. 21, 2006 (DE) 10 2006 054 734.9
Dec. 5, 2005 (DE) 10 2005 058 761.5

(51) Int. Cl. G01N 33/48

(52) U.S. Cl. 422/68.1

(57) ABSTRACT

An electrical test apparatus for testing an electrical test specimen comprising a test machine (prober), into which a contact device serving for making touching contact with the test specimen is inserted. A middle centering apparatus permits only radial temperature compensation play for the central alignment of the contact device and the test apparatus relative to one another. A corresponding method of use is disclosed.

### BACKGROUND OF THE INVENTION

The invention relates to an electrical test apparatus for the testing of an electrical test specimen, in particular a wafer, comprising a test machine (prober), into which a contact device serving for making touching contact with the test specimen is inserted/insertible.

Electrical test apparatuses of the type mentioned in the introduction serve for electrically testing an electrical test specimen, for example a wafer. For the electrical testing, a contact device is inserted into a test machine. The contact device has a multiplicity of test contacts embodied as buckling needles, for example. The free ends of the buckling needles serve for making touching contact with the test specimen. The test machine has the task of positioning the test specimen beneath the contact device (X and Y alignment) and raising the test specimen (Z positioning) in such a way that the buckling needles make touching contact with corresponding contacts of the test specimen for the test. Furthermore, the test machine has the task of producing electrical connections between the contact device and a tester. Mechanical connections may additionally be realized, if appropriate. The electrical testing of the test specimen is carried out by means of the tester, that is that electrical test circuits are established towards the test specimen in order to carry out a functional test. The test current paths run from the tester via the contact device to the test specimen, and from there back to the tester. Since the electrical test specimens are often extremely small electronic components, for example the aforementioned wafers from which individual electronic components are produced, the buckling needles have extremely small dimensions and the contact locations of the test specimen with which the buckling needles are to make contact are likewise embodied in very small size. Cameras are preferably employed for the alignment of contact device and wafer, which cameras detect the position of the wafer on a so-called test specimen carrier (chuck) of the test machine (prober) and the position of the needle tips of the buckling needles very precisely (to an accuracy of a few μm) and thereby enable a sufficiently accurate alignment of the component parts with respect to one another, so that, in the course of contact-making, the buckling needles acquire contact precisely with the test specimen contacts. Many test specimens, for example wafers, cannot generally be tested by means of a single contact-making. A plurality, often hundreds, of contact-making operations are usually necessary, that is, by means of the test specimen carrier, the test specimen is moved between the individual contact-making instances and is in each case raised for testing. In order to save test time, a complete alignment is not carried out prior to each instance of making touching contact. Rather, after the contact device has been inserted into the test machine, the position of the contact device is detected once. During the automatic testing of many test specimens, for example of wafers just constructed (for example of a batch comprising 25 items), only the positions of the wafers on the test specimen carrier are then detected, but not the position of the contact device again, that is the position of the contact tips of the buckling needles. It is assumed in this case that the positions of the needle tips of the contact device do not than change over the duration of the test process. However, the test process can last for a relatively long time, for example a few hours or even days. The test specimen, in particular a wafer, is usually temperature-regulated during the test, for example to 90° C., 150° C. or −40° C. As a result of this, the contact device is also correspondingly temperature-regulated. It goes without saying that the position of the needle tips is detected only after such a temperature-regulating operation, wherein it is possible for the latter to last a few minutes to over an hour. The problem then regularly occurs that even after the temperature-regulating operation, “movements” of the contact device still take place as a result of temperature changes. These movements may lead to instances of incorrect contact-making.

### SUMMARY OF THE INVENTION

Therefore, the invention is based on the object of providing an electrical test apparatus of the type mentioned in the introduction which enables touching contact to be made with the test specimen reliably and reproducibly, independently of the prevailing temperature.

This object is achieved according to the invention by means of a middle centering apparatus that permits only radial temperature compensation play for the central alignment of the contact device and the test machine relative to one another. This way of accommodating the contact device in the test machine crucially affects the extent of possible temperature movements, since linear changes occurring as a result of temperature compensation play proceed only from the respective center of the component parts mentioned and are present in each case, as viewed from the center; only in the radial direction. After the preferably camera assisted alignment of the centers of contact device and test machine relative to one another, the two centers remain positionally accurately positioned with respect to one another in the event of a temperature compensation play, so that no or only very small temperature-dictated linear alterations and hence position alterations take place in the region of the centers and these alterations also do not accumulate over the entire extent of the contact device, but rather, as mentioned, proceed from the center. It is ensured in this way that the test contacts of the contact device are guaranteed to make entirely satisfactory electrical contact with the associated test specimen contacts even in the event of temperature changes.

According to one development of the invention, the middle centering apparatus has at least three, and preferably four, first sliding guides arranged in a manner angularly offset with respect to one another. “Angularly offset” is to be understood to mean a circumferential positioning around the corresponding center. If three first sliding guides are used, then a determinate system is present. In the case of four first sliding guides lying angularly offset with respect to one another, an overdetermination is provided. Nevertheless, contact can be made with the test specimen reliably and reproducibly.

The first sliding guides are preferably embodied as first projection/depression guides. This means, in particular, that each of the first sliding guides is formed by a projection on the contact device or the test machine and a depression on the test machine or the contact device, which depression receives the projection with radial play and in a manner free of play in the circumferential direction. Accordingly, projection and associated depression, which may be embodied as a radial slot, in particular, can perform only radial temperature compensation plays proceeding from the center, but not temperature dictated displacements in the circumferential direction.

According to one development of the invention, it is provided that the contact device has a contact head, in particular a vertical contact head, facing the test specimen with contact elements and a wiring carrier, which has contact areas that are in touching contact with those ends of the contact elements which are remote from the test specimen. Provision is made of a middle centering device that permits only radial temperature compensation play for the central alignment of the contact head and the wiring carrier relative to one another. Consequently, alongside the middle centering apparatus between the contact device and the test machine, provision is furthermore made of a middle centering apparatus in order to provide a central alignment of the contact head and the wiring carrier relative to one another, so that there, too, temperature compensation play that occurs can only exhibit radial effects. The contact head preferably has a contact pin arrangement/contact needle arrangement having the pin-type/needle-type contact elements. In this respect, the contact pin arrangement or contact needle arrangement and the contact areas of the wiring carrier remain correspondingly aligned with one another even when exposed to different temperatures, with the result that reliable, reproducible contact-making is provided.

According to one development of the invention, the middle centering device is arranged outside the contact pin arrangement/contact needle arrangement. This configuration permits the region of the contact pin arrangement or contact needle arrangement to be kept free of the centering means, so that the region around the respective center of contact head and wiring carrier is available only for receiving pin-type contact elements, in particular needles, preferably buckling needles.

It is preferably provided that the middle centering apparatus has at least three, in particular four, second sliding guides arranged in a manner angularly offset with respect to one another. In the case of three sliding guides, the latter may lie angularly offset, in particular by 120° with respect to one another. This applies both to the first sliding guides and to the second sliding guides. As an alternative, it is possible for adjacent sliding guides to form 90° angles with respect to one another, that is that a first second sliding guide is at an angle of 90° with respect to a second second sliding guide, and the second second sliding guide is likewise at an angle of 90° with a third second sliding guide, so that the third second sliding guide lies angularly offset 180° with respect to the first second sliding guide. The same can correspondingly hold true for the first sliding guides serving for the central alignment of contact device and test machine relative to one another. As an alternative, in the case of four sliding guides, the latter may lie angularly offset in each case by 90° with respect to one another. This applies both to the first sliding guides and to the second sliding guides, the second sliding guides serving for the positioning of the contact head and the wiring carrier relative to one another.

The second sliding guides are preferably embodied as second projection/depression guides. In this case, in particular, that each of the second sliding guides may be formed by a projection on the contact head or the wiring carrier and a depression, in particular a slot, on the wiring carrier or the contact head, which depression receives the projection with radial play and in a manner free of play in the circumferential direction.

The contact device may be embodied as a test card, in particular. The test card is preferably embodied as a vertical test card, that is it has test contacts, in particular needles or buckling needles, which form an angle of 90° or approximately 90° with respect to the test plane. Test plane is to be understood to mean the contact plane, that is the interface between the test specimen and the contact head. The latter serves for making electrical touching contact with the test specimen. The test contacts preferably are embodied in pin-type/needle-type fashion and are held in the contact head displaceably in the longitudinal direction.

Finally, the invention relates to a method for testing an electrical test specimen, in particular a wafer, wherein an electrical contact device—as described above—preferably is used. In this case, the use of a test machine (prober) is provided, into which a contact device, preferably a test card, serving for making touching contact with the test specimen has been inserted. Middle centering that permits only radial temperature compensation play is effected for the central alignment of the contact device and the test machine relative to one another. Temperature compensation play in the circumferential direction is not possible.

### BRIEF DESCRIPTION OF THE DRAWINGS

The drawings illustrate the invention on the basis of exemplary embodiments, and to be precise:

FIG. 1 shows a cross section through an electrical test apparatus for the testing of an electrical test specimen,

FIGS. 2 and 3 show exemplary embodiments of a middle centering apparatus for a central alignment of contact device respectively associated with the test apparatus and test machine relative to one another,

FIG. 4 shows a first sliding guide with projection and depression, serving for the middle centering, and

FIG. 5 shows another exemplary embodiment of a projection,

FIG. 6 shows a contact device of the electrical test apparatus for making contact with the test specimen, and

FIG. 7 shows a middle centering device of the contact device for the central alignment of contact head respectively associated with the contact device and wiring carrier relative to one another.

### DESCRIPTION OF PREFERRED EMBODIMENTS

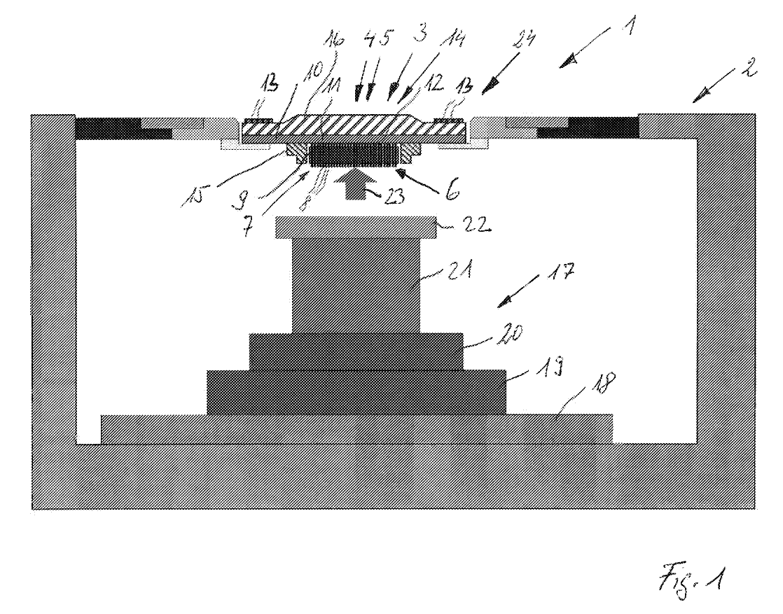

FIG. 1 shows an electrical test apparatus 1 serving for testing a test specimen, which is not revealed in FIG. 1. The test apparatus 1 has a test machine 2 (prober), into which a contact device 3 is insertible. The contact device 3 is inserted into the test machine 2 essentially horizontally and by slight lowering from above. It is embodied as a test card 4, in particular a vertical test card 5. This last means that it has a multiplicity of test contacts 7 in a contact head 6. The test contacts are embodied as needles, in particular buckling needles 8, which run transversely, in particular vertically, with respect to the preferably horizontal test plane. “Buckling needles” means that they have in each case a slight flexure, that is they deviate from a rectilinear form. The flexure may be brought about for example by holding openings of a guide 9 which lie in offset fashion and in which the buckling needles are mounted in longitudinally displaceable fashion. If the test specimen is pressed against the free ends of the buckling needles, which preferably run to a point, then the needles can spring out slightly on account of the flexure and thereby compensate for spacing irregularities and establish very good contact.

As already mentioned above, the buckling needles 8 are held in the guide 9. One of the ends of the buckling needles 8 respectively form free ends 7 for making touching contact with the test specimen. The other ends of the buckling needles 8 bear on contacts 12 of a wiring carrier 10, preferably a printed circuit board 11. The aforementioned contacts 12 of the printed circuit board 11 are connected to contacts 13 lying on the other side of the printed circuit board 11, for example via conductor tracks 41 of the printed circuit board 11. The contacts 13 are connected to a tester, which is not shown in FIG. 1 and which serves for connecting test current paths through to the test specimen in order to test the test specimen with regard to electrical functionality. Whereas the contacts 12 of the printed circuit board 11 which are touched by the buckling needles 8 lie extremely close together, the contacts 13 can be arranged in a manner distributed over a much larger area, so that the tester can be connected without any problems. A stiffening device 14, comprising a front stiffening 15 and rear stiffening 16, is provided for stiffening the contact device 3. The stiffening device 14 serves to take up the contact pressure that arises when the test specimen, as be described below, is pressed against the buckling needles 8 for the purpose of making touching contact.

The test machine 2 has a test specimen carrier 17 (chuck) having a stationary baseplate 18. Furthermore, the test specimen carrier 17 includes a Y positioning device 19, an X positioning device 20 and a Z positioning device 21. Arranged on the Z positioning device 21 is a vacuum mount 22, by means of which the test specimen can be held in a positionally invariable manner with respect to the vacuum mount 22 by vacuum. If the test specimen, for example a wafer, is then placed onto the vacuum mount 22 in planar fashion and is held by vacuum, then it can be positioned below the contact device 3 in a positionally accurate manner by means of the X and Y positioning devices 20 and 19 and with the aid of cameras in such a way that in the course of making touching contact, the buckling needles 8 make contact with corresponding contacts of the test specimen in a positionally accurate manner. For the contact-making, the Z positioning device 21 moves upward and presses the test specimen against the free ends of the buckling needles 8. This movement is indicated by means of an arrow 23 in FIG. 1.

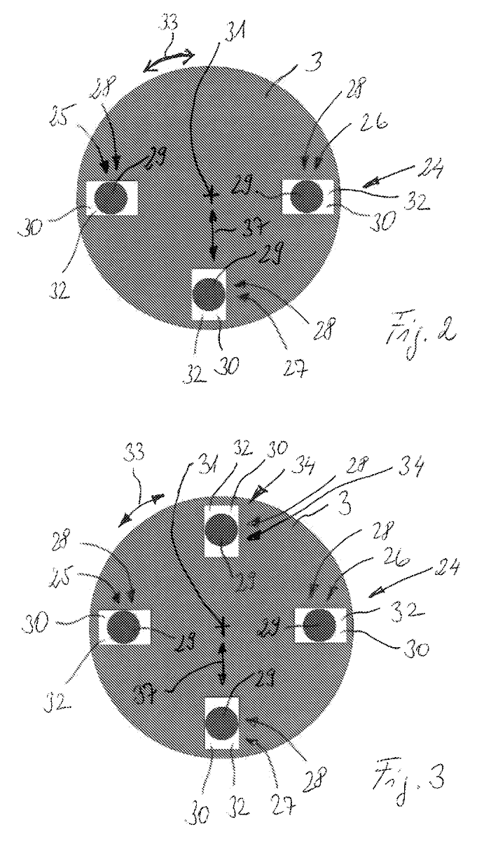

In order to hold the contact device 3 in the test machine 2 in a positionally accurate manner, a middle centering apparatus 24 is provided, which ensures that contact device 3 and test machine 2 are aligned centrally relative to one another. In this respect, reference is made to FIG. 2. The latter shows a schematic plan view of the contact device 3. The aforementioned middle centering apparatus 24 has three first radial sliding guides 25, 26 and 27 arranged in a manner angularly offset with respect to one another. The first sliding guides 25 to 27 are embodied as first projection/depression guides 28. Each first projection/depression guide has a projection 29 associated with the test machine 2. The projections 29 have for example a circular cross section and lie with radial play and, in the circumferential direction, in a manner free of play in each case in an associated radial depression 30, the depressions 30, which may also be embodied as slots and/or perforations, being embodied at the contact device 3. The first sliding guide 26 lies offset by 90° with respect to the first sliding guide 25, and the first sliding guide 27 in turn lies offset by 90° with respect to said first sliding guide 26. All the sliding guides 25 to 27 are arranged at preferably an identical distance radially around a center 31 of the contact device 3 and of the test machine 2. The depressions 30 are embodied as radially extending elongated holes 32 or correspondingly configured slots, the word “radially” referring to the center 31. The cross-sectional forms of the projections 29 are chosen in such a way that the latter lie in the elongated holes 30 in a manner free of play in the circumferential direction (double arrow 33), but in the radial direction there is play on account of the radial extent of the depressions 30 (elongated holes 32).

It ultimately becomes clear that the aforementioned middle centering apparatus 24 performs a nondisplaceable alignment in the region of the center 31 between test machine 2 and contact device 3. Even if a temperature compensation play occurs as a result of temperature change because the coefficient of thermal expansion of the test machine 2 deviates from the coefficient of thermal expansion of the contact device 3, the central alignment of contact device 3 and test machine 2 relative to one another remains since a thermal expansion play or thermal contraction play would take place only in relative fashion between the components mentioned, that is a radial displacement, which is, however, small, of the projections 29 in the depressions 30 could occur without, however, the alignment with regard to the center 31 being altered in the process. Uncontrolled stresses or an impermissibly large temperature-dictated displacement of the components are or is avoided, therefore, on account of the configuration according to the invention. This means that thermal expansions or thermal contractions of the contact device 3 relative to the test machine 2 do not lead to an alteration of the position in the middle region of the contact device 3 relative to the test machine 2. On account of this configuration, an accumulation of temperature-dictated linear changes is also restricted, since an accumulation is to be considered only from the center 31 and not over the entire diameter of the contact device 3.

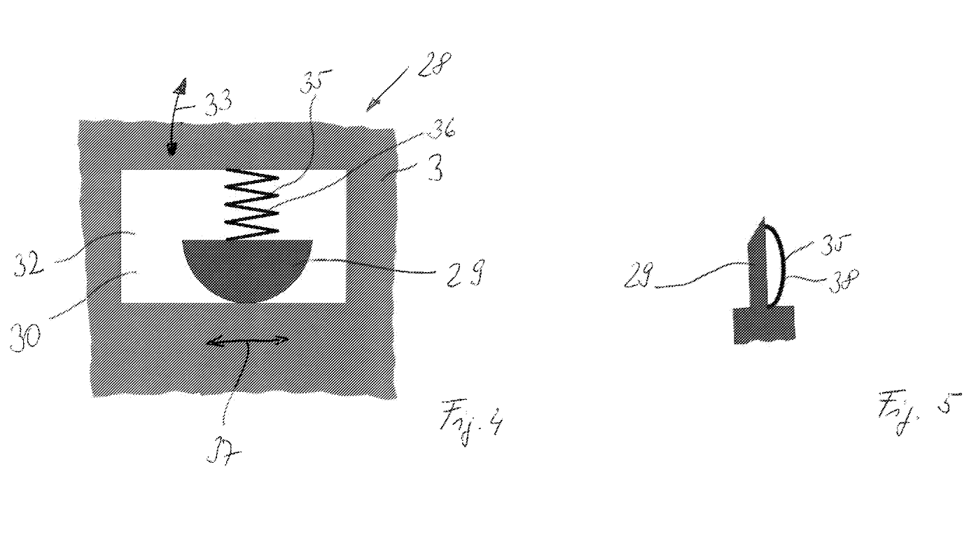

While the embodiment of FIG. 2 can be referred to as a three-slot arrangement, a four-slot arrangement is present in the embodiment of FIG. 3. The difference between the exemplary embodiment of FIG. 3 and the exemplary embodiment of FIG. 4 is that a further first radial sliding guide 34 is provided, so that the sliding guides 25, 26, 27 and 34 in each case lie offset by 90° relative to one another. For the rest, the explanations regarding FIG. 2 correspondingly hold true for FIG. 3, too. A determinate system is present in the case of the exemplary embodiment of FIG. 2, and an overdetermined system is present in the case of the exemplary embodiment of FIG. 3, but it has been shown in practice that the middle centering function is not adversely influenced even in the case of the overdetermined system. Rather, a somewhat higher stability is provided and there is compensation of unavoidable tolerances of the pin/elongated hole sliding guides 25, 26, 27 and 34 by virtue of slight bracing.

It is evident from the explanations regarding FIGS. 2 and 3 that the middle centering apparatuses 24 may also be equipped in kinematically reversed fashion, that is the projections 29 may be situated on the contact device 3 and the depressions 30 on the test machine 2. Mixed forms are also possible, that is the projections 29 are situated partly on the contact device 3 and partly on the test machine 2, and the depressions 30 are correspondingly arranged in distributed fashion.

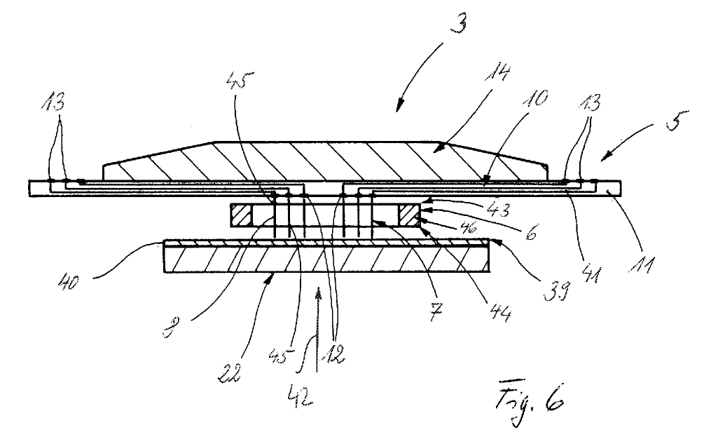

In the exemplary embodiments of FIGS. 2 and 3, the projections 29—seen in the circumferential direction (double arrow 33)—lie in the depressions 30 in a manner free of play. In this case, it must be taken into account that a very small residual play must nevertheless be present in order that the projections 29 do not become stuck in the depressions 30. In order to eliminate even this slight residual play as well, it is possible to use projections 29 in accordance with the exemplary embodiments of FIGS. 4 or 5. In the case of the exemplary embodiment of FIG. 4, the respective projection 29 has a cross-sectional configuration which bears only on one side of the depression 30. A spring 35 proceeds from the projection 29. The spring is embedded as a helical compression spring 36, in particular, and is supported on the other side of the depression 30. Complete freedom from play in the circumferential direction (double arrow 33) is obtained in this way. Play is present in the radial direction (double arrow 37), so that a relative displacement of the projection 29 and the depression 30 can be effected.

As an alternative to the embodiment of the projection 29 in FIG. 4, FIG. 5 shows a pin 29 in side view. The pin has, as spring 35, a clip spring 38 which extends at least over a partial region of the longitudinal extent of the projection 29 and can correspondingly be supported on one side of the depression 30, not illustrated in FIG. 5, like the helical compression spring 36 in FIG. 4. The clip spring 38 may also be embodied as a leaf spring.

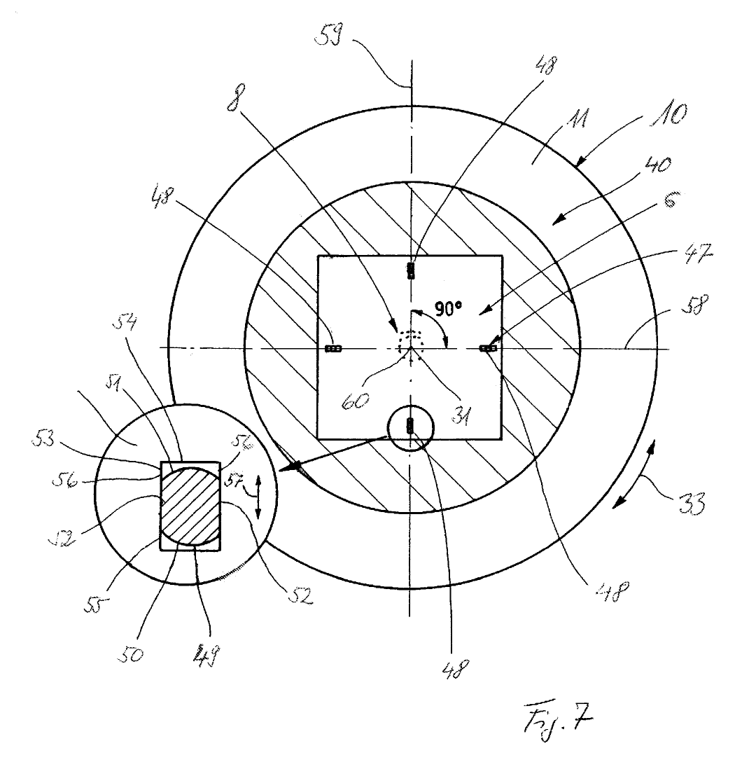

FIG. 6 shows in a schematic illustration a cross section through the contact device 3, which, for making contact with the test specimen 39, can be connected to a tester (test system) that is not illustrated by means of cable connections that are not illustrated, in order to subject the test specimen 39 to an electrical test. The test specimen 39 may be embodied in particular as a wafer 40 situated on the vacuum mount 22. During the test of the test specimen 39, the latter may be exposed to different temperatures encompassing a range of from −40° C. to +150° C., by way of example, in order to test whether the test specimen also operates entirely satisfactorily in this temperature range. For making contact with corresponding connection locations of the wafer 40, the contact device 3 is provided, which is embodied as a vertical test card 5.

The contact device 3 has the contact head 6. The contact device 3 has the stiffening device 14 for taking up contact-making forces. The contact head 6 is provided with a multiplicity of test contacts 7 which are mounted in a longitudinally displaceable fashion and which are embodied as buckling needles 8, which may also be referred to as buckling wires. The buckling needles 8 are assigned to the test specimen 39 by one of their end regions and to the wiring carrier 10 by their other end regions. The wiring carrier is embodied as a preferably multilayer printed circuit board 11 with conductor tracks 41. At their ends assigned to the contact head 6, the conductor tracks 41 have the contacts 12 assigned to the respective buckling needles 8. The wiring carrier 10 furthermore has electrical contacts 13 on its radially outer edge, which contacts can be connected to the test system (not illustrated) via the above mentioned cable connections (likewise not illustrated). The arrangement is implemented, then, in such a way that the wiring carrier 10 forms a conversion apparatus, that is the very close spacing of the tiny contacts 12 (diameter for example 50 to 300 μm) is converted into larger spacings of the contacts 13 via the conductor tracks 41. The contacts 13 have a corresponding size in order to be able to produce the connection to the cables of the tester.

During the testing of the test specimen 39, the latter moves—in a manner supported by the stiffening device 14—in the axial direction (arrow 42), the vacuum mount 22 holding the test specimen 39, toward the contact head 6, so that the end faces of test contacts 7 impinge on the test specimen 39, on the one hand, and on the contacts 12, on the other hand. Since the test contacts 7 are embodied as buckling needles 8, that is they are configured such that they are slightly resilient by virtue of flexure in the axial direction, entirely satisfactory contact-making is possible. The contact head 6 has for example two parallel ceramic plates 43 and 44 which lie at a distance from one another and are provided with bearing holes 45 for receiving the buckling needles 9. The parallel spaced-apart position of the two ceramic plates 43 and 44 is realized by means of a spacer 46.

Provided between the contact head 6 and the wiring carrier 10 is a middle centering device 47 (FIG. 7) having four second radial sliding guides 48 lying offset at an angular distance of 90° with respect to one another in the circumferential direction (double arrow 33). FIG. 7 reveals that the test specimen 39, that is the wafer 40, is embodied as a circular plate. It has integrated circuits that are not illustrated, by way of example. In order to electrically test the circuits, the contact head 6, which is preferably embodied such that it is square in plan, with its buckling needles 8, which are not revealed specifically in FIG. 7, is caused to make touching contact with the wafer 40 multiply in different positions in order to be able to test in each case a corresponding region of the wafer 40. A circular or rectangular plan is also conceivable, of course. The second sliding guide 48 lying at the bottom in FIG. 7 is depicted as an enlarged detail. It has a projection 49 in the form of a profiled pin 50 projecting from the contact head 6 in the axial direction (in the direction of the arrow 42 in FIG. 6). The profiled pin 50 has two mutually diametrically opposite, parallel, planar guide surfaces 52 on its outer lateral surface 51. The profiled pin 50 extends with its free end right into a depression 53 formed on the wiring carrier 10, that is the printed circuit board 11. The depression 53 is preferably configured as a perforation 54 in the printed circuit board 11. The depression 53 has a radial elongated hole form, that is constitutes an elongated hole 55. The depression 53 has two depression walls 56 which run parallel to one another and are spaced apart from one another in such a way that they receive the guide surfaces 52 of the profiled pin 50 essentially in a manner free of play. The longitudinal extent of the elongated hole 55 is made larger than the corresponding longitudinal dimension of the projection 49, so that relative movement in the direction of the depicted double arrow 57 can take place between the contact head 6 and the wiring carrier 10, that is the printed circuit board 11, in accordance with the enlarged illustration in FIG. 7. A relative movement transversely thereto is not possible since this is prevented by the guidance of the guide surfaces 52 on the depression walls 56. As an alternative to the arrangement illustrated, the profiled pin 50 may project from the printed circuit board 11 and the depression 53 may be embodied in the contact head 6.

It becomes clear from FIG. 7 that four second sliding guides 48 are arranged in such a way that they lie on two imaginary radial lines 59 and 59 that intersect at an angle of 90°, the radial lines 58 and 59 intersecting at a midpoint 60 and the midpoint 60 forming the center 31 of the contact device 3. The buckling needles 8 are arranged around the center 31. The four second sliding guides 48 are situated radially outwardly from the buckling needles 8, the longitudinal extents of the elongated holes 55 being oriented in such a way that they in each case lie centrally on the radial lines 58 and 59. The guide surfaces 52 of the individual profiled pins 50 are configured according to the elongated hole alignment of the elongated holes 55.

It ultimately becomes clear that in the event of a material expansion or material contraction arising from temperature exposure, the components contact head 6 and wiring carrier 10 are fixed with respect to one another in the region of the center 31 on account of the middle centering device 47 and relative movements can be effected only in the direction of the radial lines 58 and 59. This ensures that the above mentioned linear changes resulting from different coefficients of thermal expansion of the materials used for the components cannot have the effect that offset paths occur which are so large that the end faces of the buckling needles 8 that are assigned to the printed circuit board 11 no loner impinge on the contact areas of the contacts 12. The middle centering on account of the middle centering device 47 prevents such large offset paths, since the linear changes that occur begin proceeding from the center 31 and thus lie symmetrically with respect to the middle and therefore, as seen in the radial direction, are only half as large as an offset which, if the middle centering device were not used, might occur if outer buckling needles 8 are seated centrally onto assigned contacts 12, so that the diammetrically opposite, likewise outer buckling needles 8 lead to faulty contacts on account of the cumulating ear expansions or linear contractions.

1. An electrical test apparatus for testing of an electrical test specimen, comprising
a test machine (prober), into which a contact device serving for making touching contact with the test specimen is inserted;
a middle centering apparatus connected between the contact device and the test machine and operable to permit only radial temperature compensation play for causing central alignment of the contact device and the test machine relative to one another.

2. The test apparatus according to claim 1, wherein the middle centering apparatus comprises at least three, first sliding guides arranged angularly offset with respect to one another around a center.

3. The test apparatus according to claim 2, wherein the first sliding guides comprise first projection/depression guides.

4. The test apparatus according to claim 3, wherein each of the first sliding guides is formed by a projection on one of the contact device and the test machine and a depression on the other of the test machine and the contact device, wherein the depression receives the projection with radial play but free of play in a circumferential direction.

5. The test apparatus according to claim 1, wherein the contact device further comprises a contact head including contact elements and a wiring carrier, the wiring carrier has contact areas in touching contact with first ends of the contact elements which are remote from the test specimen and the middle centering apparatus permits only radial temperature compensation play for the central alignment of the contact head and the wiring carrier relative to one another.

6. The test apparatus according to claim 1, wherein the contact elements of the contact head form a contact pin arrangement/contact needle arrangement and are embodied in a pin-type/needle-type fashion.

7. The test apparatus according to claim 6, wherein the middle centering device is arranged outside the contact pin arrangement/contact needle arrangement.

8. The test apparatus according to claim 1, wherein the middle centering apparatus includes at least three, second sliding guides arranged angularly offset with respect to one another.

9. The test apparatus according to claim 8, wherein the second sliding guides comprise second projection/depression guides.

10. The test apparatus according to claim 9, wherein each of the second sliding guides comprises a projection on one of the contact head and the wiring carrier and a respective depression on the other of the wiring carrier and the contact head, wherein the depression receives the projection with radial play and free of play in the circumferential direction.

11. The test apparatus according to claim 1, wherein the contact device is a test card.

12. The test apparatus according to claim 11, wherein the test card is a vertical test card.

13. The test apparatus according to claim 1, wherein the contact head is operable for making electrical touching contact.

14. The test apparatus according to claim 1, wherein the contact head includes test contacts embodied as needles.

15. A method for testing an electrical test specimen, by means of an electrical test apparatus, the method comprising providing a test machine (prober); inserting a contact device serving for making touching contact with the test specimen into the test machine, and providing a middle centering that permits only radial temperature compensation play and permitting only the radical temperature compensation play of the contact device and the test apparatus being for central alignment of the contact device and the test apparatus relative to one another.
